(19) United States
(12) Patent Application Publication
(10) Pub. No.: US 2005/0171493 A1
(43) Pub. Date: Aug. 4, 2005

(54) Brush

(76) Inventor: Anthony C. Nicholls, Liss (GB)

Correspondence Address: Jane Massey Licata;Licata & Tyrrell P.C., 66 E. Main Street, Marlton, NJ, 08053, US

(21) Appl. No.: 10/773,706

(22) Filed: Feb. 6, 2004

(30) Foreign Application Priority Data
Feb. 2, 2004 (GB) GB0402249.7

(51) Int. Cl. A61M 25/00

(52) U.S. Cl. 604/267; 15/159.1

(57) ABSTRACT

A system of selecting a brush for use in brusing a catheter lumina.

The present invention relates to brushes primarily for use in brushing catheter lumina. This application claims the benefit of priority to the United Kingdom application GB0402249.7 filed Feb. 2, 2004.

The use of catheters is an essential part of daily medical practice. However, catheterisation is an extremely invasive technique carrying a relatively high rate of complication with approximately 30% of insertions bringing additional morbidity to the patient. Once successfully inserted, complications such as infection and occlusion can result in the replacement of the catheter in 40% of patients during the period of therapeutic need.

Studies over the past 30 years have shown that as many as 80% of catheters which are suspected to be the cause of infection and subsequently replaced, are, in fact, replaced unnecessarily. UK (EP) Patent No. 0 792 375 describes an assay method which employs an endoluminal brush to dislodge debris from an in situ catheter lumen. This method allows the assessment of catheter infection without the need to remove the catheter.

Brushes of the type described in UK (EP) Patent No. 0 792 375 have been routinely used to obtain samples from the inner lumen of catheters. These samples may be subjected to laboratory analysis in order to provide an accurate assessment of catheter colonisation and a predictive diagnosis of catheter related sepsis.

Whilst the assessment of catheter colonization and the predictive diagnosis of catheter related sepsis has been improved, the treatment of catheter occlusion has remained poor. The use of chemical agents to dissolve occluding materials has had limited short term effect but most occlusion is still resolved by the replacement of the catheter with its attendant complication rate. As a consequence of their use in sampling, brushes of the type described in UK (EP) Patent No. 0 792 375 have been shown to effectively remove debris from the inner lumen of the catheter, improving flow rates and extending the useful life of the catheter. However, such use has been hindered by inherent fears of potential complications, such as the risk of embolism, where small particles detach from the catheter and then lodge in the vessels of the lungs or brain and cause extensive tissue damage, or damage to the catheter itself.

In this regard, when obtaining samples for colonization evaluation, the possibility of discharging material into the body is considered to be small and of little clinical consequence. However, using an endoluminal brush on an occluded catheter greatly increases the possibility that obstructing material, which may also be infected, will be discharged into the patient's body, in particular, into the central venous circulatory system. Such discharge may then give rise to additional morbidity in the form of pulmonary embolism, stroke, sub-acute bacterial endocarditis or sepsis. Furthermore, the act of brushing can dislodge material that the brush fails to retrieve, leaving such material to be discharged to the body following the subsequent use of the catheter.

It is therefore an object of the present invention to overcome such problems.

According to a first aspect of the present invention, there is provided a system of selecting a brush for use in brushing a catheter lumen, the system comprising:—providing a plurality of brushes of varying brush head diameters, the bristles of each respective brush head being of substantially the same length; identifying a catheter lumen to be brushed and determining its widest cross-sectional internal dimension from along its length; selecting a brush from said plurality of brushes; wherein the ratio of the brush head diameter of the selected brush with respect to the widest cross-sectional internal dimension of the catheter lumen is greater than 1.0.

Preferably, the ratio of the head diameter of the selected brush to the widest cross-sectional internal dimension of the catheter lumen is greater than 1.2.

Further preferably, the ratio of the head diameter of the selected brush to the widest cross-sectional internal dimension of the catheter lumen is within the range 1.25 to 2.5.

In this connection, it has been found that in order to prevent pushing material in front of the brush upon insertion thereof into the catheter lumen, the brush must have a brush head which has a greater diameter than the widest cross-sectional internal dimension of the catheter lumen. In this way, during insertion of the brush, the bristles orientate themselves towards the proximal end of the catheter, i.e. rearwardly, in the opposite direction from the forward insertion direction of the brush.

The ratio of the brush head diameter to the widest cross-sectional internal dimension of the catheter lumen is also relevant with regard to the retention capacity of debris within the bristles. The wider the catheter lumen, the greater the potential volume of debris present therein. In this respect, as the length of the bristles is increased relative to the internal dimensions of the catheter lumen, the effective density of bristles within the catheter lumen is also increased thereby increasing the retention capacity of debris within the bristles. It is advantageous to increase the density of bristles by increasing their length as opposed to increasing the brush head wind length, i.e. the length of the brush head, because as described below, an increase in brush head wind length will limit the extent to which the brush head can navigate around a curved path.

Further, the ratio of the brush head diameter to the widest cross-sectional internal dimension of the catheter lumen is relevant in the prevention of catheter puncture. When the bristles are compressed within the catheter lumen, they act as a variable “spacer” ensuring that the wire stem of the brush is kept substantially in the center of the catheter lumen and is not allowed to come into contact with the wall of the catheter. As the diameter of the lumen increases, additional compression is necessary to ensure centralization of the wire and its smooth passage within the catheter lumen.

Accordingly, there is preferably a non-linear relationship between the rate of increase of the widest cross-sectional internal dimension of the catheter lumen and the head diameter of the brush. More preferably, the rate of increase of brush head diameter is greater than that of the widest cross-sectional internal dimension of the catheter lumen.

For catheter lumina with a widest cross-sectional internal dimension of between 0.6 to 2.0 mm, the ratio of the head diameter of the selected brush to the widest cross-sectional internal dimension of the catheter lumen is preferably within the range 1.25 to 1.7.

For catheter lumina with a widest cross-sectional internal dimension of between 2.1 to 2.4 mm, the ratio of the head diameter of the selected brush to the widest cross-sectional internal dimension of the catheter lumen is conveniently within the range 1.45 to 1.7.

For catheter lumina with a widest cross-sectional internal dimension of between 2.5 to 3.0 mm, the ratio of the head diameter of the selected brush to the widest cross-sectional internal dimension of the catheter lumen is preferably within the range 2.0 to 2.4.

For catheter lumina with a widest cross-sectional internal dimension of between 3.2 to 4.0 mm, the ratio of the head diameter of the selected brush to the widest cross-sectional internal dimension of the catheter lumen may be within the range 2.0 to 2.5.

In preferred embodiments, each of said plurality of brushes has a brush head front end, the bristles of each brush head being configured to compress inwardly and rearwardly as the brush is inserted forwardly into a catheter lumen, the majority of the bristles remaining in a rearwardly orientated direction as the brush is retracted from the catheter lumen.

As the bristles compress inwardly they are sufficiently resilient to glide over debris attached to the walls of the catheter lumen, allowing the brush to be passaged to the end of the catheter, without pushing material ahead of it. Thereafter, upon withdrawal of the brush, a majority of the bristles are arranged to remain orientated rearwardly towards the proximal end of the catheter, and so remove debris from the lumen walls of the catheter upon withdrawal of the brush.

In this regard, if the brush head diameter is the same as the internal dimension of the catheter lumen, the bristles will produce too much friction with the walls of the catheter lumen, thus making it difficult to pass the brush along the lumen, in addition to the brush dislodging debris during insertion.

The bristles at a front end of the brush head may however be arranged so that they reverse in orientation on withdrawal of the brush from the catheter lumen. In this regard, as explained above, the majority of the bristles remain rearwardly orientated so that they dig in, scour out and trap debris in the catheter lumen. In contrast, the bristles at the front end of the brush head, face forwardly such that as the brush is extracted from the catheter lumen, they trap or smooth out any debris that has not been caught by the rearwardly orientated bristles. In so doing, any such debris, if not caught by the front or rear ends of the brush head, will at worst, be re-applied to the lumen wall and will not thereby increase the risk of embolisms. Further, between the rearwardly orientated bristles and forwardly orientated bristles there is formed a reservoir for accumulating dislodged debris which has not been caught by the rearwardly orientated bristles. Such a reservoir can be particularly useful should the rear end of the brush become particularly clogged with debris.

Each brush may comprise a longitudinally extending wire with the brush head formed at one end of the wire, wherein the brush head comprises laterally extending bristles forming a bristle head.

In preferred embodiments, the wire is of greater length than the catheter lumen. Preferably, the length of the brush is limited by bending the wire at a point of required length from the terminal end of the bristle head.

The wire, therefore, physically marks an end-stop which dictates the distance the brush can be inserted into the catheter. Preferably, the bristles are formed of nylon.

In preferred embodiments, the gauge of the wire is greater for short straight catheter lumina compared to the gauge for long, curved catheter lumina. The lower gauge wire allows the brush to be extended along curved routes of insertion. Additionally, the gauge of the wire in long catheters is sufficiently low to allow the wire to buckle preventing the brush from being forced through the wall of the catheter, thus obviating possible puncture wounds.

Further preferably, the brush has a shorter wind length for long curved catheter lumina compared to short straight catheter lumina. The shorter wind length therefore accommodates the need for the brush to pass around curves within the catheter insertion route.

Preferably, the bristles are arranged in a rear orientated spiral. Such a spiral orientation increases the effectiveness of the brush in removing debris from the catheter lumen, by rifling the lumen during withdrawal of the brush.

Preferably still, the wire within the brush head is relatively rigid. Therefore, the integrity of the bristle head is maintained during insertion and withdrawal of the brush.

The brush may be provided with a sheath for connection to the proximal opening of the catheter. Further, the sheath may be connected to the catheter by a luer lock connector.

Therefore, as the brush is withdrawn from the catheter the debris can be collected into the sheath, and the bristle head cut from the wire and isolated within the sheath for examination of the debris.

According to a second aspect of the present invention, there is provided a method of removing debris from a catheter lumen, the method comprising; (I) inserting a brush into the catheter lumen, (ii) extending the brush towards the distal opening of the catheter lumen and (iii) withdrawing the brush from the catheter lumen; wherein, whilst extending the brush forwardly into the catheter lumen, the bristles are urged rearwardly in the opposite direction from the motion of the brush so that they can glide over, and do not substantially dislodge, the debris from the catheter lumen, and during withdrawal of the brush, a majority of the bristles remain rearwardly orientated in the direction of withdrawal so that the debris is dislodged from the catheter lumen.

Preferably, a portion of the brush is extended beyond the distal opening of the catheter. Further preferably, 2.0 mm of the brush is extended beyond the distal opening of the catheter. Therefore, in preferred embodiments, the bristles which extend beyond the distal opening of the catheter become forwardly orientated during withdrawal of the brush.

Preferably, in such an arrangement, a reservoir is formed between the rearwardly orientated bristles and the forwardly orientated bristles, and the reservoir can accumulate dislodged debris which is not embedded within the rearwardly orientated bristles, and the forwardly orientated bristles act to retain dislodged debris which is not caught by the reservoir.

Conveniently, on withdrawal of the brush from the lumen, the forwardly orientated bristles smooth the surface of the catheter lumen. By smoothing the surface of the catheter lumen, the cross-sectional area of the catheter lumen is maximized, thereby increasing the fluid capacity further.

In this connection, if the catheter lumen is restricted by as little as 5% of its cross-sectional diameter, this will result in a 33% reduction in flow rate. It is, therefore, important to maximize the cross-sectional area of the catheter lumen.

Additionally, by extending a portion of the brush beyond the distal opening of the catheter, any debris attached to the distal opening of the catheter can be removed. In this connection, the accumulation of debris which may then form an occlusion within a catheter lumen can commence at the distal opening of the catheter.

Alternatively, the brush is extended to no closer than 2.0 cm to the distal opening of the catheter. In this respect, when removing a sample of infected debris from a catheter lumen, it is important not to spread the infection to the distal opening of the catheter. An infection within a catheter lumen will progress from the proximal end towards the distal opening of the catheter, and, therefore, there is no need to extend the brush all the way to the distal opening of the catheter.

Preferably, the insertion length of the brush is limited by bending the wire of the brush at a point of required length from the terminal end of the bristle head, prior to insertion of the brush into the catheter lumen. The wire, therefore, physically marks an end-stop which dictates the distance the brush can be inserted into the catheter.

Conveniently, the dislodged debris is embedded and retained within the bristles. Further conveniently, once the bristles are full with the debris, the bristle head forms a plug for removing debris not embedded within the bristles.

Accordingly, since the debris either becomes embedded within the bristles or is removed by the plug forming bristle head, the amount of debris removed from the catheter is maximized.

In preferred embodiments, after withdrawal of the brush, fluid is withdrawn from the lumen to remove any residual loose debris. Therefore, the possibility of discharging any loose debris into the patient upon re-use of the catheter for further infusion therapy is greatly reduced.

Conveniently, the catheter is in situ. Further conveniently, the brush is maintained within an enclosed system during insertion and withdrawal of the brush. Therefore, the user and patient are protected from infection.

In a further embodiment of the present invention, the catheter lumen is treated with a chemical agent to at least partially loosen the debris within the catheter lumen. Preferably, the catheter lumen is treated with a solution of alcohol prior to insertion of the brush into the catheter lumen. By partially loosening the debris within the catheter lumen prior to insertion of the brush, the amount of debris removed from the catheter lumen can be further maximized.

The brush of the present invention is therefore able to retrieve the bulk of the material contained within the lumen on the reverse traverse and to remove any remaining free floating debris prior to re-connection.

Accordingly, the ability of a brush to restore the patency of a catheter, i.e. the ability of the catheter to allow the free flow of therapeutic materials into the patient or the retrieval of body fluid samples from the catheter, is improved by the present invention.

Further, the brush of the present invention prevents, or significantly minimizes, the risk of any morbid consequence of brushing.

According to a further aspect of the present invention there is provided a brush for use in brushing a catheter lumen; the brush having a head of bristles, the bristles having substantially the same length along the brush head's length; wherein the ratio of the head diameter of the brush to the widest cross-sectional internal dimension of the catheter lumen is greater than 1.0.

Preferably, the ratio of the head diameter of the brush to the widest cross-sectional internal dimension of the catheter lumen is greater than 1.2.

Further preferably, the ratio of the head diameter of the selected brush to the widest cross-sectional internal dimension of the catheter lumen is within the range 1.25 to 2.5.

In this connection, there is preferably provided a brush for use in brushing a catheter lumen; the brush having a head of bristles, the bristles having substantially the same length; wherein if the widest cross-sectional internal dimension of the catheter lumen is between 0.6 to 2.0 mm, the ratio of the head diameter of the brush to the widest cross-sectional internal dimension of the catheter lumen is within the range 1.25 to 1.7; or wherein if the widest cross-sectional internal dimension of the catheter lumen is between 2.1 to 2.4 mm, the ratio of the head diameter of the brush to the widest cross-sectional internal dimension of the catheter lumen is within the range 1.45 to 1.7; or wherein if the widest cross-sectional internal dimension of the catheter lumen is between 2.5 to 3.0 mm, the ratio of the head diameter of the brush to the widest cross-sectional internal dimension of the catheter lumen is within the range 2.0 to 2.4; or wherein if the widest cross-sectional internal dimension of the catheter lumen is between 3.2 to 4.0 mm, the ratio of the head diameter of the brush to the widest cross-sectional internal dimension of the catheter lumen is within the range 2.0 to 2.5.

Accordingly, there is preferably a non-linear relationship between the rate of increase of the widest cross-sectional internal dimension of the catheter lumen and the head diameter of the brush. More preferably, the rate of increase of brush head diameter is greater than that of the widest cross-sectional internal dimension of the catheter lumen.

Conveniently, the brush head has a front end and a rear end, wherein during insertion of the brush into a catheter lumen in a forward direction, the bristles on both the front and rear end orientate themselves rearwardly, whilst on withdrawal of the brush from the catheter lumen in a rearward direction, the bristles of the front end orientate themselves in a forward direction.

Conveniently, there is provided a set of brushes for use in brushing a catheter lumen, the set comprising a plurality of brushes as hereinbefore described, the set having brushes of different brush head diameter.

Preferably, the set of brushes is provided together with a plurality of catheters and indication means for selecting an appropriate brush/catheter combination. Such indication means could include suitable markings to ensure correct matching of the catheter lumen and brush.

Preferably, there is further provided a brush as hereinbefore described, the brush being provided together with an appropriate catheter.

According to a further aspect of the invention there is provided a method for treating an occluded catheter lumen situated in a patient, the method comprising the steps: (a) inserting a brush having bristles into a proximal opening of the catheter lumen, the catheter lumen containing debris; (b) extending the brush towards a distal opening of the catheter lumen, and (c) withdrawing the brush from the catheter lumen; wherein, whilst extending the brush forward into the catheter lumen, the bristles are urged rearwardly in the opposite direction from the motion of the brush so that they can glide over, and do not dislodge, the debris from the catheter lumen, and during withdrawal of the brush, a majority of the bristles remain rearwardly orientated in the direction of withdrawal so that the debris is dislodged from the catheter lumen.

Preferably, the brush is extended just beyond the distal opening, for example up to 3 mm beyond. More conveniently, the brush is extended 2 mm beyond the distal opening.

Alternatively, in certain preferred embodiments, the brush is extended no closer that 2.0 cm to the distal opening.

Preferably, before step (a) the catheter is treated with a chemical agent to at least partially loosen the debris.

Moreover, before step (c) the catheter may be treated with a chemical agent to at least partially loosen the debris.

The patient may be placed in a supine position before step (a).

In a preferred embodiment, after step (c), up to 6 catheter volumes of fluid are withdrawn from the catheter lumen. More preferably, after step (c), up to 4 to 5 catheter volumes of fluid are withdrawn from the catheter lumen. The catheter volumes of fluid are preferably withdrawn using a syringe of greater than 10 cc.

An example of the present invention will now be described in detail with reference to the drawings in which;

FIG. 1 shows a brush of the present invention before entry into a catheter lumen;

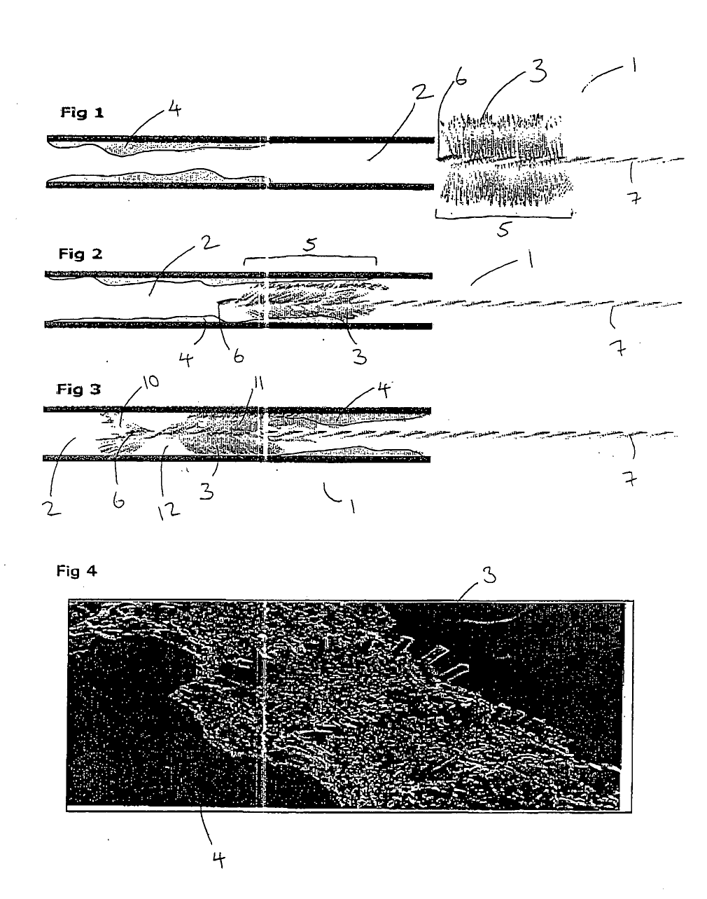

FIG. 2 shows the brush of FIG. 1 being pushed forward within the catheter lumen;

FIG. 3 shows the brush of FIG. 1 being withdrawn within the catheter lumen;

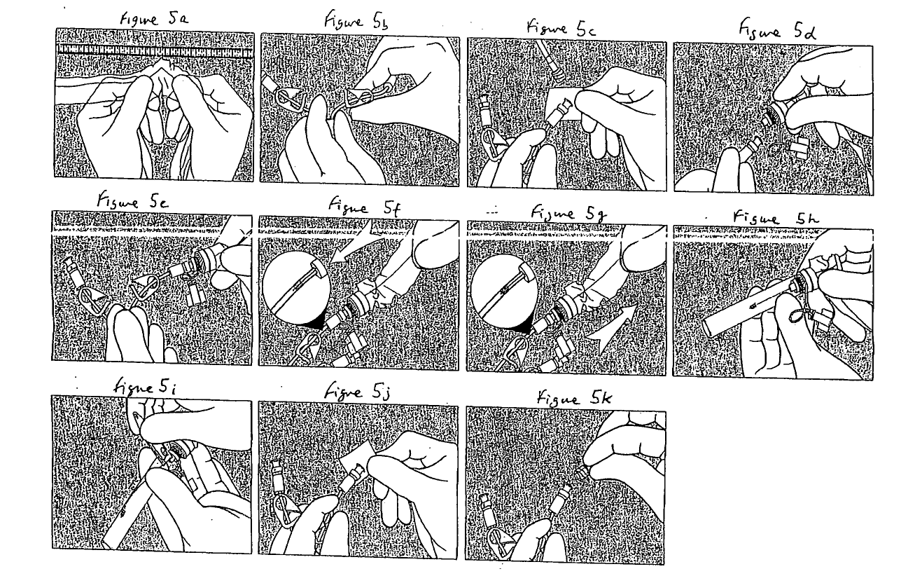

FIG. 4 shows a scanning Electron micrograph of bristles 3 of a brush of the present invention embedded with catheter debris 4; and

FIGS. 5a to 5k show a suggested procedure for using the brush of FIG. 1.

FIGS. 1 to 3 hence show a brush 1 of the present invention before being introduced into a catheter lumen 2, being pushed into the lumen and being withdrawn from the lumen.

In this regard, it has been found that in order to prevent pushing debris in front of the brush 1, the brush 1 must have a bristle head 5 which is of greater diameter than that of the catheter lumen 2. The relationship between the catheter lumen diameter and the bristle head diameter is given by a ratio which increases with lumen diameter. The ratio has preferred minimum and maximum values as shown in Table 1 below. In this connection, the critical dimension of the lumen is the widest cross sectional dimension, which for a circular catheter is of course the diameter, whereas for an oval or D-shaped lumen, the widest cross sectional dimension needs to be determined separately. For any particular catheter lumen, it is imperative to select the correct brush and no one brush will be suitable for all catheter lumina.

TABLE 1
The relationship between catheter lumen
diameter and bristle head diameter.

| Critical Lumen | | | |
|---|---|---|---|
| Dimension | | | Bristle Head |
| (mm) | Minimum Ratio | Maximum Ratio | diameter (mm) |
| 0.6 to 0.8 | 1.25 | 1.7 | 1.0 |
| 0.9 to 1.2 | 1.25 | 1.7 | 1.5 |
| 1.5 to 2.0 | 1.25 | 1.7 | 2.5 |
| 2.1 to 2.4 | 1.45 | 1.7 | 3.5 |
| 2.5 to 3.0 | 2.0 | 2.4 | 6.0 |
| 3.2 to 4.0 | 2.0 | 2.5 | 8.0 |

As shown in FIG. 2, with a diameter greater than that of the catheter lumen, on entering the catheter lumen 2, the brush head 5 is compressed such that all of the bristles 3 are oriented rearwardly to the proximal end of the catheter in the opposite direction to the forward motion of the brush. In such an orientation, the bristles 3 do not brush but “glide” over the surface of the debris 4 within the catheter lumen 2, without dislodging debris from the catheter lumen. The crushed head of the brush 1 acts as a nylon “bearing” allowing the easy passage of the brush 1 through the catheter lumen 2 which is lubricated by the debris 4 on the inner wall. The brush is advanced such that the tip of the brush head 6 extends 2.0 mm beyond the distal opening of the catheter. As shown in FIG. 3, in reverse motion the majority of the bristles 3 of the brush 1 are oriented rearwardly in the direction of motion and effectively brush the debris 4 from the walls and luminal space of the catheter. The disturbed debris is embedded within the bristles 3 and retained by the bristle head 5. Once engorged, the bristle head 5 of the brush 1 acts like a piston removing additional debris in the proximal end of the lumen. In this respect, the brush head has a front end 10 and a rear end 11, wherein during insertion of the brush into a catheter lumen in a forward direction, the bristles on both the front and rear end orientate themselves rearwardly, whilst on withdrawal of the brush from the catheter lumen in a rearward direction, the bristles of the front end which extended beyond the distal opening of the catheter during insertion orientate themselves in a forward direction. As the brush is extracted from the catheter lumen, the bristles of the front end trap or smooth out any debris that has not be caught by the rearwardly orientated bristles. In so doing, any such debris, if not caught by the front end of the brush head, will at worst, be re-applied to the wall of the catheter and will not thereby increase the risk of embolisms. Further, between the rearwardly orientated bristles and forwardly orientated bristles there is formed a reservoir 12 for catching dislodged debris which has not been caught by the rearwardly orientated bristles.

As the brush 1 is withdrawn, the debris 4 is displaced into a sheath (not shown) which surrounds the brush 1 and which remains outside the catheter attached to its hub by a secure luer lock connector (not shown). By displacing the debris into a sheath, attached to the catheter by a secure luer lock connector, the brushing procedure can remain in an enclosed system, thus protecting the user and patient from infection. The bristle head can then be cut from the wire 7 and isolated for examination of the entrapped debris 4, shown in FIG. 4.

FIG. 4 also shows the bristles 3 of the bristle head 5 arranged in a rear-orientated spiral. This arrangement increases the effectiveness of the brush 1 in removing debris from the catheter lumen 2, since it effectively “rifles” the catheter lumen 2 whilst being withdrawn.

Following completion of the brushing procedure the brush is disconnected from the hub of the catheter and replaced by a 10 ml syringe. 10 ml of fluid is withdrawn from the lumen, effectively removing any loose debris and, therefore, avoiding the possibility of discharging such debris into the patient upon reuse of the catheter for further infusion therapy.

Not only is the dimension of the brush relevant in the prevention of removal of debris from the catheter walls during insertion of the brush but also insomuch as the dimension also dictates the bristle capacity available for the retention of dislodged debris. The bigger the lumen then the greater the potential volume of debris present and this factor, rather than the need to avoid forward brushing, dictates the ratio of brush head diameter to lumen diameter.

The ratio between critical dimension and head diameter is also relevant to the prevention of catheter puncture. As the bristles are compressed, they act as a variable “spacer” ensuring that the wire stem of the brush is kept in the center of the lumen and is not allowed to come into contact with the wall of the catheter. As the diameter of the lumen increases additional compression is necessary to ensure smooth passage of the wire. Hence, as the lumen diameter increases, so does the ratio between critical dimension and head diameter Additional safeguard against puncture is afforded by controlling the strength of the wire. The gauge of the wire used to manufacture any particular brush is such that it will “buckle” and not allow further forward motion if it comes into contact with solid material such as the wall of the catheter. Wire gauge is defined according to the nature of the material being used in the manufacture of the target catheter, its wall thickness and the likely tortuosity of its insertion pathway. Brushes for catheters that have a straight and relatively short route of insertion, such as internal jugular polyurethane, will have greater wire gauge than those designed for long catheters likely to have curved routes of insertion such as subclavian silicon. Similarly brushes designed for curved routes will have a shorter wind length. Wind length is defined as the length of the brush head, i.e. the length of the brush which has bristles. The shorter wind length accommodates the need for the brush to pass around curves within the catheter insertion route. Since the wire is rigid throughout the bristle wind, the length of this wind and the diameter of the lumen will determine the degree of curvature that might be traversed by the brush head.

The utility of the brush is maximized by its passage towards the tip of the catheter. In order to facilitate this the length of the catheter which is to be inserted to the catheter is measured. The correct brush, which should have a wire length greater than that of the catheter, is selected. Using the required insertion length of the catheter, and adding a known length for the dimension of the luer connector, generally 2.0 mm, a figure is derived for the required length of insertion. Using a measure such as a tape provided with the brush, the length is marked on the flexible wire of the brush by physically bending the wire. This prevents any insertion of excess wire into the catheter and provides an easily detected point of reference at which to start removing the brush. In this connection, if 2.0 mm of the brush is to extend beyond the distal opening of the catheter, the user need only mark a length on the wire corresponding to the length of the catheter, since the dimension of the luer connector will account for the additional 2.0 mm required.

By using a brush having the correct bristle head diameter and length, in combination with the correct gauge of wire, an occluded catheter can be successfully cleared of debris and returned to normal function with reduced fear of embolization or damage to the catheter.

Additionally, because the causative organisms associated with catheter-related blood stream infection are resident within the debris contained in the catheter lumen, brushing also removes infection and effectively treats colonization of causative organisms within the catheter. Such treatment is effected by the mechanical removal of infecting organisms. During such treatment, the brush should be extended to no closer than 2.0 cm to the distal opening of the catheter. In this respect, when removing a sample of infected debris from a catheter lumen, it is important not to spread the infection to the distal opening of the catheter. An infection within a catheter lumen will progress from the proximal end towards the distal opening of the catheter, and, therefore, there is no need to extend the brush all the way to the distal opening of the catheter.

Further, because it is known that the deposition of “biofilm”, a matrix of bacterial growth and a protective slime, encourages the build up of debris and the colonization of a catheter as a precursor to blood stream infection, the routine brushing of catheters acts to prevent both occlusion and infection.

The present invention further encompasses a set of brushes for use in brushing a catheter lumen. The set may in this regard comprise a plurality of brushes, of varying brush head diameter, with a suitable indication means such that a user can readily match a catheter with an appropriate brush. This may, for example, take the form of simple color coordinated or otherwise readily distinguishable markings on catheters and brushes.

A suggested procedure for carrying out the invention will now be described with reference to FIGS. 5a to 5k.

Suggested Procedure

Materials Required

The brushing kit contains a self-enclosed sterile Brush, a medical drape, a measuring device, a test tube, a cap and Instructions for Use, inclusive of a Compatibility Table.
(1) Brush Kit Note: use one Endoluminal Brush Kit per lumen being sampled.
(2) Sterile syringe (at least 10 cc)
(3) Cleaning agent.
(4) Sterile gloves, mask and other materials required to ensure that a sterile environment is created when manipulating the catheter.
(5) Scissors or equivalent sterile device to clip the wire once sampling is completed.
   Brushing Procedure

Brushing may be performed at the bedside and does not require the use of an operating or surgical room.
(1) Provide a sterile operative field according to standard hospital policy or procedures for manipulating indwelling catheters. The use of sterile gloves, gowns, face masks and drapes is recommended.
(2) Check to ensure that the correct Brush, appropriate for the catheter lumen being examined has been selected.

(3) Calculate the maximum length of the Brush to be inserted into the catheter. If removing potentially infected debris from the catheter lumen for sampling only, measure from the catheter hub to within 2 cm of the catheter tip as outlined below

| | | |
|---|---|---|
| (a)Length of catheter lumen (hub to tip): | | cm |
| (b)Add 1.5 cm for the length of the luer lock collar: | +1.5 | cm |
| (c)Subtract 2 cm to ensure Brush does not go to the | −2.0 | cm |
| catheter tip | | |
| (d)Total maximum length of flexible wire required: | | cm |

If measuring for use in clearing an occluded catheter lumen, then replace step (c) above with:—(c) add 2 mm to ensure that the brush extends just beyond the catheter tip.
(4) Using the measuring device, measure the flexible wire from the tip (which starts immediately within the luer lock collar) to the length calculated above, 3 (d). Kink the wire at this point to clearly mark the maximum length of Brush insertion. (See FIG. 5a).
(5) To prevent inadvertent air aspiration during Brush insertion, the patient should be placed in a supine position.
(6) Place a sterile drape under the catheter hub.
(7) Occlude the catheter lumen that is being brushed with a plastic line clip. (See FIG. 5b).
(8) Disconnect the giving set from the selected lumen hub.
(9) Swab the hub with the recommended cleaning solution and allow the hub to dry. (See FIG. 5c).
(10) Connect the luer lock of the Brush assembly to the catheter hub. (See FIG. 5d).
(11) Release the line clip. (See FIG. 5e).
(12) Advance the Brush through the lumen of the catheter, towards the catheter tip. Advance small sections of the flexible wire, through the catheter hub, with smooth, regular pushes and controlled pressure from a point as close to the hub as possible. Continue to insert the Brush until the Brush is at the desired insertion position, which is indicated by the kink in the wire. (See FIG. 5f) DO NOT attempt to force the Brush past a difficult obstruction.
   NOTE: Resistance may be felt as the Brush passes through the manifold of a multi-lumen catheter, but this will reduce when the Brush enters the catheter lumen. Depending on the catheter design, the restrictions of triple lumen manifolds may prevent the Brush from advancing through the manifold and into the proximal and medial lumens. If this occurs remove, proceed to Step 13, but record that the brush sampled the HUB ONLY.
(13) Fully withdraw the Brush into the plastic sheath. (See FIG. 5g)
   CAUTIONS: (1) The Brush should not be re-advanced once the withdrawal from the lumen has begun. (2) Blood may be removed with a bio-film sample. Due to the risk of exposure to HIV (Human Immunodeficiency Virus) or other blood borne pathogens, health care workers should routinely use universal Blood and body-fluid precautions in the care of all patients. (3) In the unlikely event that the Brush becomes lodged in the catheter and consistent, steady pressure fails to effect withdrawal, it is advised that the catheter be removed with the brush in place and the Brush retrieved from the Catheter outside of the vein.
(14) Occlude the catheter using the line clip.
(15) Disconnect the sheath from the catheter hub and seal the sheath with the luer cap, or clip (with sterile scissors) the Brush and approximately 6 cm of wire into the test tube or equivalent sterile container. (See FIG. 5I).
(16) Using standard hospital procedures for removing blood withdraw 4-5 catheter volumes of blood from the brushed lumen.
   NOTE: This procedure is a preventive measure to ensure that any loosened fibrin, not removed by the Brush, is withdrawn.
(17) Re-swab the hub using the recommended cleaning solution. (See FIG. 5j).
(18) Reconnect the line to the original infusion set or cap appropriately. (See FIG. 5k).
(19) Label the Brush sheath or the sterile container containing the Brush and the blood sample, which was withdrawn, immediately post-brushing.
(20) Send the sample to the laboratory for analysis.
   NOTE: The Brush, blood sample and the container should be disposed of accordingly to the hospital's approved procedure for clinical waste.

The above suggested procedure can be carried out by a medical professional in a medical facility. However, the method and apparatus also encompasses self brushing procedures carried out by non-medical professionals such as the patient themselves or their partners, in for example, their own homes.

The invention encompasses apparatus and a method for removing debris from a catheter lumen and also apparatus and a method for determining potential infections present in the catheter lumen. In this regard, samples of catheter lumen debris obtained at step 15 of the suggested procedure identified above can be assayed for the latter purpose.

1. A system of selecting a brush for use in brushing a catheter lumen, the system comprising:
providing a plurality of brushes of varying brush head diameters, the bristles of each respective brush head being of substantially the same length along the brush head's length;
identifying a catheter lumen to be brushed and determining its widest cross-sectional internal dimension from along its length;
selecting a brush from said plurality of brushes;
wherein the ratio of the head diameter of the selected brush to the widest cross-sectional internal dimension of the catheter lumen is greater than 1.0.

2. A system according to claim 1, wherein the ratio of the head diameter of the selected brush to the widest cross-sectional internal dimension of the catheter lumen is greater than 1.2.

3. A system according to claim 2, wherein the ratio of the head diameter of the selected brush to the widest cross-sectional internal dimension of the catheter lumen is within the range 1.25 to 2.5.

4. A system according to claim 1, wherein there is a non-linear relationship between the rate of increase of the widest cross-sectional internal dimension of the catheter lumen and the head diameter of the brush.

5. A system according to claim 4, wherein the rate of increase of brush head diameter is greater than that of the widest cross-sectional internal dimension of the catheter lumen.

6. A system according to claim 1, wherein for catheter lumina with a widest cross-sectional internal dimension of between 0.6 to 2.0 mm, the ratio of the head diameter of the selected brush to the widest cross-sectional internal dimension of the catheter lumen is within the range 1.25 to 1.7.

7. A system according to claim 1, wherein for catheter lumina with a widest cross-sectional internal dimension of between 2.1 to 2.4 mm, the ratio of the head diameter of the selected brush to the widest cross-sectional internal dimension of the catheter lumen is within the range 1.45 to 1.7.

8. A system according to claim 1, wherein for catheter lumina with a widest cross-sectional internal dimension of between 2.5 to 3.0 mm, the ratio of the head diameter of the selected brush to the widest cross-sectional internal dimension of the catheter lumen is within the range 2.0 to 2.4.

9. A system according to claim 1, wherein for catheter lumina with a widest cross-sectional internal dimension of between 3.2 to 4.0 mm, the ratio of the head diameter of the selected brush to the widest cross-sectional internal dimension of the catheter lumen is within the range 2.0 to 2.5.

10. A system according to claim 1, wherein each of said plurality of brushes has a brush head front end, the bristles of each brush head being configured to compress inwardly and rearwardly as the brush is inserted forwardly into a catheter lumen, the majority of the bristles remaining in a rearwardly orientated direction as the brush is retracted from the catheter lumen.

11. A system according to claim 10, wherein each brush comprises a longitudinally extending wire with the brush head formed at one end of the wire, wherein the brush head comprises laterally extending bristles forming a bristle head.

12. A system according to claim 11, wherein the wire is of greater length than the catheter lumen.

13. A system according to claim 11, wherein the length of the brush is limited by a user bending the wire at a point of required length from the terminal end of the bristle head.

14. A system according to claim 1, wherein the bristles are formed of nylon.

15. A system according to claim 11, wherein the gauge of the wire is greater for short straight catheter lumen compared to the gauge for long, curved catheter lumen.

16. A system according to claim 11, wherein the brush has a shorter wind length for long curved catheter lumen compared to short straight catheter lumen.

17. A system according to claim 1, wherein the bristles are arranged in a rear orientated spiral.

18. A system according to claim 1, wherein a sheath is provided for connection to a proximal opening of the catheter.

19. A system according to claim 18, wherein the sheath is connected to the catheter lumen by a luer lock connector.

20. A method of removing debris from a catheter lumen, the method comprising;
(a) inserting a brush having bristles into an opening in the catheter lumen,
(b) extending the brush to a distal opening of the catheter lumen, and
(c) withdrawing the brush from the catheter lumen; wherein,
whilst extending the brush forward into the catheter lumen, the bristles are urged rearwardly in the opposite direction from the motion of the brush so that they can glide over, and do not dislodge, the debris from the catheter lumen, and
during withdrawal of the brush, a majority of the bristles remain rearwardly orientated in the direction of withdrawal so that the debris is dislodged from the catheter lumen.

21. A method according to claim 20, wherein the dislodged debris is embedded and retained within the bristles.

22. A method according to claim 20 or 21, wherein a portion of the brush is extended beyond the distal opening of the catheter.

23. A method according to claim 20, wherein 2.0 mm of the brush is extended beyond the distal opening of the catheter.

24. A method according to claim 22, wherein the bristles which extend beyond the distal opening of the catheter become forwardly orientated during withdrawal of the brush.

25. A method according to claim 24, wherein, during withdrawal of the brush, a reservoir is formed between the rearwardly orientated bristles and the forwardly orientated bristles, and the reservoir can accumulate dislodged debris which is not embedded within the rearwardly orientated bristles, and the forwardly orientated bristles act to retain dislodged debris which is not caught by the reservoir.

26. A method according to claim 24, wherein, on withdrawal of the brush from the lumen, the forwardly orientated bristles smooth the surface of the catheter lumen.

27. A method according to claim 20, wherein the brush is extended to no closer than 2.0 cm to the distal opening of the catheter.

28. A method according to claim 20, wherein an insertion length of the brush is limited to the required length by a user bending the wire of the brush at a point of such length from the terminal end of the bristle head, prior to insertion of the brush into the catheter lumen.

29. A method according to claim 20, wherein, once the bristles are full with the debris, the bristle head forms a plug for removing any additional debris not embedded within the bristles.

30. A method according to claim 20, wherein after withdrawal of the brush, fluid is withdrawn from the lumen to remove any residual loose debris.

31. A method according to claim 20, wherein the catheter is in situ in a patient.

32. A method according to claim 20, wherein, prior to insertion of the brush into the catheter lumen, the catheter lumen is treated with a chemical agent to at least partially loosen the debris within the catheter lumen.

33. A method according to claim 32, wherein the catheter lumen is treated with a solution of alcohol prior to insertion of the brush into the catheter lumen.

34. A brush for use in the system of claim 1.

35. A brush for use in brushing a catheter lumen; the brush having a head of bristles, the bristles having substantially the same length along the brush head's length;
wherein the ratio of the head diameter of the brush to the widest cross-sectional internal dimension of the catheter lumen is greater than 1.0.

36. A brush according to claim 35, wherein the ratio of the head diameter of the brush to the widest cross-sectional internal dimension of the catheter lumen is greater than 1.2.

37. A brush according to claim 35, wherein the ratio of the head diameter of the brush to the widest cross-sectional internal dimension of the catheter lumen is in the range 1.25 to 2.5.

38. A brush according to any one of claim 35, wherein there is a non-linear relationship between the rate of increase of the widest cross-sectional internal dimension of the catheter lumen and the head diameter of the brush.

39. A brush according to claim 38, wherein the rate of increase of brush head diameter is greater than that of the widest cross-sectional internal dimension of the catheter lumen.

40. A brush according to claim 35;
wherein if the widest cross-sectional internal dimension of the catheter lumen is between 0.6 to 2.0 mm, the ratio of the head diameter of the brush to the widest cross-sectional internal dimension of the catheter lumen is within the range 1.25 to 1.7; or
wherein if the widest cross-sectional internal dimension of the catheter lumen is between 2.1 to 2.4 mm, the ratio of the head diameter of the brush to the widest cross-sectional internal dimension of the catheter lumen is within the range 1.45 to 1.7; or
wherein if the widest cross-sectional internal dimension of the catheter lumen is between 2.5 to 3.0 mm, the ratio of the head diameter of the brush to the widest cross-sectional internal dimension of the catheter lumen is within the range 2.0 to 2.4; or
wherein if the widest cross-sectional internal dimension of the catheter lumen is between 3.2 to 4.0 mm, the ratio of the head diameter of the brush to the widest cross-sectional internal dimension of the catheter lumen is within the range 2.0 to 2.5.

41. A brush according to claim 35, wherein the brush head has a front end and a rear end, wherein during insertion of the brush into a catheter lumen in a forward direction, the bristles on both the front and rear end orientate themselves rearwardly, whilst on withdrawal of the brush from the catheter lumen in a rearward direction, the bristles of the front end orientate themselves in a forwardly direction.

42. A set of brushes for use in brushing a catheter lumen the set comprising a plurality of brushes according to claim 35, the set having brushes of different brush head diameter.

43. A set of brushes according to claim 42, the set being provided together with a plurality of catheters and indication means for selecting an appropriate brush/catheter combination.

44. A brush according to claim 35, the brush being provided together with an appropriate catheter.

45. A method for treating an occluded catheter lumen situated in a patient, the method comprising the steps:
(a) inserting a brush having bristles into a proximal opening of the catheter lumen, the catheter lumen containing debris;
(b) extending the brush towards a distal opening of the catheter lumen, and
(c) withdrawing the brush from the catheter lumen; wherein,
whilst extending the brush forward into the catheter lumen, the bristles are urged rearwardly in the opposite direction from the motion of the brush so that they can glide over, and do not dislodge, the debris from the catheter lumen, and
during withdrawal of the brush, a majority of the bristles remain rearwardly orientated in the direction of withdrawal so that the debris is dislodged from the catheter lumen.

46. A method according to claim 45, wherein the brush is extended up to 3 mm beyond the distal opening.

48. A method according to claim 45, wherein the brush is extended no closer that 2.0 cm to the distal opening.

49. A method according to claim 45, wherein before step (a) the catheter is treated with a chemical agent to at least partially loosen the debris.

50. A method according to claim 45, wherein before step (c) the catheter is treated with a chemical agent to at least partially loosen the debris.

51. A method according to claim 45, wherein the patient is placed in a supine position before step (a).

52. A method according to claim 45, wherein after step (c), up to 6 catheter volumes of fluid are withdrawn from the catheter lumen.

53. A method according to claim 52, wherein after step (c), up to 4 to 5 catheter volumes of fluid are withdrawn from the catheter lumen.

54. A method according to claim 52, wherein the catheter volumes of fluid are withdrawn using a syringe of greater than 10 cc.
